(19) United States
(12) Patent Application Publication
(10) Pub. No.: US 2024/0033400 A1
(43) Pub. Date: Feb. 1, 2024

(54) COLLAGEN IV BIOINKS

(71) Applicants: THE UNIVERSITY OF SYDNEY, Sydney, NSW (AU); SOUTH EASTERN SYDNEY LOCAL HEALTH DISTRICT, Caringbah, NSW (AU); THE UNIVERSITY OF WOLLONGONG, Gwynneville, NSW (AU)

(72) Inventors: Jingjing YOU, Ryde, NSW (AU); Gerard SUTTON, Chatswood, NSW (AU); Morgan OVERMASS, North Willoughby, NSW (AU); Frank LOVICU, Sydney, NSW (AU); Gordon WALLACE, Gwynneville, NSW (AU)

(21) Appl. No.: 18/550,870

(22) PCT Filed: Mar. 18, 2022
(86) PCT No.: PCT/AU2022/050247

§ 371 Date: Sep. 15, 2023

(30) Foreign Application Priority Data
Mar. 18, 2021 (AU) 2021900794

(51) Int. Cl. A61L 27/24; A61L 27/36; A61L 27/38

(52) U.S. Cl. CPC A61L 27/24; A61L 27/3687; A61L 27/3813; A61L 27/3808

(57) ABSTRACT

The present invention relates to compositions suitable for the delivery of agents to biological targets such as tissues and cells and/or capable of promoting the growth and/or proliferation of cells, and methods for the production thereof. Specifically, a printable collagen bioink comprising type IV collagen is used to produce crosslinked collagen gels comprising mammalian cells, such as endothelial or epithelial cells, for corneal regeneration.

### INCORPORATION BY CROSS-REFERENCE

This application claims priority from Australian provisional patent application number 2021900794, filed on 18 Mar. 2021, the entire contents of which are incorporated herein by cross-reference.

### TECHNICAL FIELD

The present invention relates generally to the fields of biology and medicine. More specifically, the present invention relates to compositions suitable for the delivery of agents to biological targets such as tissues and cells and/or capable of promoting the growth and/or proliferation of cells, and methods for the production thereof.

### BACKGROUND

In cases of trauma to biological tissue or in instances where disease results in tissue degeneration, the traditional approach is to attempt to reconstruct said tissue with a transplant from a suitable donor. More recently, bioengineering has afforded an alternative means for the replacement of biological tissue. Bioengineering allows for the creation of synthetic substitute tissue prior to transplantation. Additionally or alternatively, cell-based therapies include the injection of cultured cells with appropriate growth factors, thereby allowing for tissue regeneration in situ. The latter method has the benefit of technical simplicity and is particularly useful for the restoration of function when the shape of the biological tissue under repair is crucial, for example, corneal tissue.

The cornea is the clear component of the protective covering of the eye. It allows light to pass through the pupil and is the primary refractive element of the eye's optical system. The cornea consists of five layers: the outer epithelium, Bowman's layer, the stroma, Descemet's membrane, and the inner endothelium. The corneal stroma accounts for approximately 90% of the overall thickness of the cornea and is mostly made up of collagen.

Corneal blindness is the second major cause of blindness worldwide. Causes of corneal blindness include diseases such as trachoma, onchocerciasis, leprosy, ophthalmia neonatorum, and xerophthalmia, and other processes such as ocular trauma, corneal ulceration, and complications arising from the use of traditional eye medicines.

Corneal injuries represent the most common ophthalmic emergency presentation in Australia and approximately 75% of all cases are due to the presence of foreign bodies or abrasions in the cornea. These injuries alone are estimated to cost the Australian population more than $155 million per year and, if not treated effectively, can lead to infection and scarring, resulting in permanently impaired vision.

In mild cases, a damaged cornea is able to regenerate via normal healing pathways. In other cases, however, the cornea's normal healing mechanism is insufficient, leading to the formation of non-healing defects which can result in corneal melting, corneal neovascularisation, loss of transparency, infection, scarring and diminished vision to the point of blindness.

Corneal endothelial disease is one example of a condition affecting the cornea which would benefit from improved compositions and methods for tissue repair and/or regeneration. Whilst the surgical treatment of corneal endothelial disease has evolved in recent years through the use of lamellar transplants, this surgery remains technically difficult and utilises precious donor corneal tissue. Recognized complications include subluxation of the donor tissue.

Current medical treatments for corneal injuries include antibiotics, eye pads, sutures and surgical glues, which may help with minor issues. However, they do not adequately address issues arising in more advanced situations including pain relief, infection and/or the development of scar tissue. Infection represents a significant complication and often requires hospitalisation. Scarring, which is common in severe corneal injuries, can lead to permanent vision loss. In such cases, corneal transplantation is the only option for visual rehabilitation, but a shortage of donor corneas exists worldwide.

Many of the aforementioned issues are not restricted to corneal injuries and disease, and also prevail in the case of damage and/or deterioration of other body tissues.

A need exists for improved compositions and methods for tissue repair and/or regeneration, and/or for the effective delivery of agents to biological targets such as tissues and cells.

### SUMMARY OF THE INVENTION

The present invention alleviates at least one of the problems associated with current compositions and/or methods for tissue repair and/or regeneration and the delivery of agents to biological targets. In the context of the cornea, the present inventors have surprisingly found that a bioink comprising type IV collagen is compatible with cells, e.g. lens epithelial cells, and/or supports the growth and/or proliferation of corneal endothelial cells. Additionally, the bioink of the present invention is transparent, crosslinkable and/or printable and may be used to create gels and/or scaffolds of varying sizes due to properties such as mechanical strength and flexibility.

Further, the bioink of the present invention may support the delivery of a range of agents to biological targets such as tissues and cells, extending its application to a broad range of targets beyond those related to the cornea.

Without limitation, the compositions and methods described herein are generally useful for the delivery of agents (e.g. cells, drugs and/or or other substances) to biological targets (e.g. tissue, membranes, cells) and may find application, for example, in tissue repair and/or regeneration, including corneal tissue.

The present invention relates at least in part to the following embodiments:
Embodiment 1. A composition comprising:
   6-24 mg/ml type IV collagen;
   0.04-0.15 M sodium ions and/or 0.008-0.4 M calcium ions; and
   one or more crosslinking agents.
Embodiment 2. The composition of embodiment 1, wherein the composition comprises 0.01-0.1 mg riboflavin.
Embodiment 3. The composition of embodiment 1 or embodiment 2, wherein the one or more crosslinking agents are capable of activation by light.
Embodiment 4. The composition of embodiment 3, wherein the light is UV light, blue light, green light or white light.
Embodiment 5. The composition of embodiment 1, wherein the composition comprises fibrinogen and/or thrombin.
Embodiment 6. The composition of any one of embodiments 1 to 5, wherein the composition further comprises mammalian cells.
Embodiment 7. The composition of embodiment 6, wherein the mammalian cells comprise or consist of human cells.
Embodiment 8. The composition of embodiment 6 or embodiment 7, wherein the mammalian cells comprise any one or more of: neuronal cells, epithelial cells, photoreceptor cells, müller cells, endothelial cells.
Embodiment 9. The composition of any one of embodiments 6 to 8, wherein the mammalian cells comprise or consist of epithelial cells and/or endothelial cells.
Embodiment 10. The composition of embodiment 8 or embodiment 9, wherein the epithelial cells are lens epithelial cells.
Embodiment 11. The composition of embodiment 8 or embodiment 9, wherein the endothelial cells are corneal endothelial cells.
Embodiment 12. The composition of any one of embodiments 1 to 11, further comprising any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.
Embodiment 13. The composition of embodiment 12, wherein the composition comprises a culture medium comprising the ions and amino acids.
Embodiment 14. The composition of embodiment 12 or embodiment 13, wherein:
(i) the growth factors comprise vascular endothelial growth factor (VEGF) and/or fibroblast growth factor (FGF); and/or
(ii) the vitamins comprise riboflavin; and/or
(iii) the matrix proteins comprise type I collagen; and/or
(iv) the matrix proteins comprise laminin.
Embodiment 15. The composition of any one of embodiments 1 to 14, wherein the ions are components of an ionic salt included in the composition.
Embodiment 16. The composition of any one of embodiments 1 to 15, wherein the type IV collagen is neutralised.
Embodiment 17. The composition of any one of embodiments 1 to 16, wherein the composition comprises:
(i) 6-24 mg/ml type IV collagen, 0.06-0.1 M sodium ions, and 0.01-0.05 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.
Embodiment 18. The composition of any one of embodiments 1 to 17, wherein the composition comprises:
(i) less than 24 mg/ml type IV collagen;
(ii) more than 0.04 M sodium ions; and
(iii) more than 0.008 M calcium ions.
Embodiment 19. A method of preparing a composition, the method comprising:
(i) providing a solution comprising:
   6-24 mg/ml type IV collagen;
   one or more crosslinking agents; and
   0.04-0.15 M sodium ions; and/or 0.008-0.4 M calcium ions;
(ii) applying the solution to a surface; and
(iii) activating the one or more crosslinking agents.
Embodiment 20. The method of embodiment 19, wherein applying the solution to a surface forms a layer, and wherein steps (ii) and (iii) are repeated a plurality of times, wherein each layer is applied on top of the preceding layer.
Embodiment 21. The method of embodiment 19 or embodiment 20, wherein the one or more crosslinking agents comprise 0.01-0.1 mg riboflavin.
Embodiment 22. The method of any one of embodiments 19 to 21, wherein the activating in step (iii) comprises applying light capable of activating the one or more crosslinking agents.
Embodiment 23. The method of embodiment 22, wherein the light comprises UV light, blue light, green light or white light.
Embodiment 24. A method of preparing a composition, the method comprising:
(i) providing a solution comprising:
   6-24 mg/ml type IV collagen;
   one or more crosslinking agents; and
   0.04-0.15 M sodium ions; and/or 0.008-0.4 M calcium ions; and
(ii) applying the solution to a surface, wherein the solution is divided into at least two components prior to applying to the surface.
Embodiment 25. The method of embodiment 24, further comprising the steps of:
(iii) adding fibrinogen to at least one component to form formulation (a);
(iv) adding thrombin to at least one component to form formulation (b); and
(v) combining formulations (a) and (b) to form a gel.
Embodiment 26. The method of any one of embodiments 19 to 25, further comprising adding mammalian cells to the solution and/or the composition.
Embodiment 27. The method of embodiment 26, wherein the mammalian cells comprise or consist of human cells.
Embodiment 28. The method of embodiment 26 or embodiment 27, wherein the mammalian cells comprise any one or more of: neuronal cells, epithelial cells, photoreceptor cells, müller cells, endothelial cells.
Embodiment 29. The method of any one of embodiments 26 to 28, wherein the mammalian cells comprise or consist of epithelial cells and/or endothelial cells.
Embodiment 30. The method of embodiment 28 or embodiment 29, wherein the epithelial cells are lens epithelial cells.
Embodiment 31. The method of embodiment 28 or embodiment 29, wherein the endothelial cells are corneal endothelial cells.
Embodiment 32. The method of any one of embodiments 19 to 31, wherein the solution further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.
Embodiment 33. The method of embodiment 32, wherein the solution comprises a culture medium comprising the ions and amino acids.
Embodiment 34. The method of embodiment 32 or embodiment 33, wherein:
(i) the growth factors comprise vascular endothelial growth factor (VEGF) and/or fibroblast growth factor (FGF); and/or
(ii) the vitamins comprise riboflavin; and/or
(iii) the matrix proteins comprise type I collagen; and/or
(iv) the matrix proteins comprise laminin.
Embodiment 35. The method of any one of embodiments 19 to 34, wherein the ions are components of an ionic salt included in the mixture.
Embodiment 36. The method of any one of embodiments 19 to 35, wherein the type IV collagen is neutralised.
Embodiment 37. A composition obtained or obtainable by the method of any one of embodiments 19 to 36.
Embodiment 38. A method of sealing the surface of tissue, the method comprising applying the composition of any one of embodiments 1 to 18 or embodiment 37 to the tissue.
Embodiment 39. A method of delivering agents to tissue, the method comprising applying the composition of any one of embodiments 1 to 18 or embodiment 37 to the tissue.
Embodiment 40. A method of culturing cells, the method comprising applying the cells to the composition of any one of embodiments 1 to 18 or embodiment 37.
Embodiment 41. The method of embodiment 40, wherein the cells comprise or consist of epithelial cells and/or endothelial cells.
Embodiment 42. The method of embodiment 41, wherein the epithelial cells are lens epithelial cells.
Embodiment 43. The method of embodiment 41, wherein the endothelial cells are corneal endothelial cells.
Embodiment 44. A composition of any one of embodiments 1 to 18 or embodiment 37 for use in sealing the surface of tissue.
Embodiment 45. A composition of any one of embodiments 1 to 18 or embodiment 37 for use in delivering agents to tissue.
Embodiment 46. A composition of any one of embodiments 1 to 18 or embodiment 37 for use in culturing cells.
Embodiment 47. The use of embodiment 46, wherein the cells comprise or consist of epithelial cells and/or endothelial cells.
Embodiment 48. The use of embodiment 47, wherein the epithelial cells are lens epithelial cells.
Embodiment 49. The use of embodiment 47, wherein the endothelial cells are corneal endothelial cells.
Embodiment 50. A kit, package or device for preparing a composition, the kit comprising
   6-24 mg/ml of type IV collagen;
   0.04-0.15 M of sodium ions and/or 0.008-0.4 M of calcium ions; and
   one or more crosslinking agents.
Embodiment 51. Use of a kit, package or device comprising type IV collagen, sodium ions, calcium ions, and one or more crosslinking agents, for preparing a composition comprising:
   6-24 mg/ml of the type IV collagen;
   0.04-0.15 M of the sodium ions and/or 0.008-0.4 M of the calcium ions; and
   the one or more crosslinking agents.
Embodiment 52. The kit, package or device of embodiment 50 or the use of embodiment 51, wherein the composition comprises 0.01-0.1 mg riboflavin.
Embodiment 53. The kit, package or device of embodiment 50 or embodiment 52 or use of embodiment 51 or embodiment 52, wherein the one or more crosslinking agents are capable of activation by light.
Embodiment 54. The kit, package or device or the use of embodiment 53, wherein the light comprises UV light, blue light, green light or white light.
Embodiment 55. The kit, package or device of any one of embodiments 50 or 52 to 54 or the use of any one of embodiments 51 to 54, wherein the composition further comprises epithelial cells and/or endothelial cells.
Embodiment 56. The kit, package or device or the use of embodiment 55, wherein the epithelial cells are lens epithelial cells.
Embodiment 57. The kit, package or device or the use of embodiment 55, wherein the endothelial cells are corneal endothelial cells.
Embodiment 58. The kit, package or device of any one of embodiments 50 or 52 to 57 or the use of any one of embodiments 51 to 57, wherein the composition further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.

### Definitions

As used in this application, the singular form “a”, “an” and “the” include plural references unless the context clearly dictates otherwise. For example, the term “component” also includes a plurality of components.

As used herein, the term “comprising” means “including”. Variations of the word “comprising”, such as “comprise” and “comprises,” have correspondingly varied meanings. Thus, for example, a composition “comprising” component ‘A’ may consist exclusively of component ‘A’ or may include one or more additional components (e.g. component ‘B’ and/or component ‘C’).

As used herein, the term “subject” includes any animal of economic, social or research importance including bovine, equine, ovine, primate, avian and rodent species.

Hence, a “subject” may be a mammal such as, for example, a human, or a non-human mammal.

As used herein, the term “tissue” will be understood to encompass both cells that are component/s of the tissue and organ/s formed from the tissue.

As used herein, the term “kit” refers to any delivery system for delivering materials. Such delivery systems include systems that allow for the storage, transport, or delivery of reaction reagents (e.g., labels, reference samples, supporting material, etc. in appropriate containers) and/or supporting materials (e.g., buffers, written instructions for performing an assay etc.) from one location to another. For example, kits may include one or more enclosures, such as boxes, containing the relevant reaction reagents and/or supporting materials.

As used herein, the term “about” when used in reference to a recited numerical value includes the recited numerical value and numerical values within plus or minus ten percent of the recited value.

As used herein, the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any numerical value derivable therein, and any range derivable therein.

As used herein, the term “between” when used in reference to a range of numerical values encompasses the numerical values at each endpoint of the range. For example, calcium ions with a concentration of between 0.008 M and 0.4 M is inclusive of calcium ions with a concentration 0.008 M and calcium ions with a concentration 0.4 M.

As used herein, the term “more than” when used in reference to a numerical value will be understood to mean “greater than or equal to”. For example, more than 0.018 M calcium ions encompasses a concentration of 0.018 M calcium ions and all concentrations of calcium ions greater than 0.018 M.

As used herein, the term “less than” when used in reference to a numerical value will be understood to mean “less than or equal to”. For example, less than 15 mg/ml type IV collagen encompasses a concentration of 15 mg/ml type IV collagen and all concentrations of type IV collagen less than 15 mg/ml.

As used herein, the term “neutralised” when used to describe type IV collagen will be understood to mean that the pH of the collagen solution is between 6.7 and 7.6. For example, “neutralised” type IV collagen could have a pH between 6.8 and 7.5, or between 6.9 and 7.4, or between 7.0 and 7.3, or between 6.9 and 7.2, etc.

As used herein, the term “culturing”, when used in the context of cells, will be understood to mean promoting the growth and/or proliferation of cells. Variations of the word “culturing”, such as “culture” and “cultures,” have correspondingly varied meanings. Thus, for example, a method of “culturing” cells will be understood to mean a method of promoting an increase in both the size and number of cells.

As used herein, the term “crosslink”, when used in reference to collagen, will be understood to mean an increase in intra- and intermolecular covalent bonds. Variations of the word “crosslink”, such as “crosslinked” and “crosslinking,” have correspondingly varied meanings. In some instances, collagen “crosslinking” will be understood to mean an increase in intra- and interfibrillar covalent bonds in collagen fibrils.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### BRIEF DESCRIPTION OF THE FIGURES

Preferred embodiments of the present invention will now be described by way of example only, with reference to the accompanying figures wherein:

FIG. 1 provides graphs demonstrating rheological test data of col-4 solutions exposed to UV light (1A) or blue light (1B) for 3 minutes starting from the 3 minute point. FIG. 1A provides the results of a UV rheological test of col-4 solution (12 mg/mL) showing the gelation point as determined by G′ and G″. FIG. 1B provides the results of a rheological test of col-4 solution (12 mg/mL) when exposed to blue light (400-500 nm) showing the gelation point as determined by G′ and G″.

FIG. 2 provides representative images of a col-4 printed lattice using optimised printing parameters such as temperature, speed, flow rate and tip diameter.

FIG. 3 provides a representative diagram of the process used to generate a flat col-4 membrane on a coverslip. FIG. 3C provides an example of a set-up used with paper substituted for the coverslips used as stands in FIG. 3B, demonstrating another method of varying height.

FIG. 4 provides representative images of different types of col-4 scaffolds. FIG. 4A shows a thin film (50 μm) created on a glass coverslip which could be used to culture cells and create corneal endothelial sheets. FIG. 4B shows a thick gel (5 mm).

FIG. 5 provides representative images which show differences in transparency between col-1 and col-4 biomaterials seen both macroscopically (top) and microscopically (bottom).

FIG. 6 provides representative images demonstrating the effect of col-1 and col-4 thick (1 mm) gels and thin (50 μm) films on the morphology of lens epithelial cells following 1 week of exposure. α-SMA (red) is a marker for elongated actin stress fibres characteristic of myofibroblastic cells, cells that have undergone an epithelial-to-mesenchymal (EMT) from epithelial cells. These elongated myofibroblastic cells appear to be mainly present in col-1 conditions while col-4 conditions retain their epithelial phenotype highlighted by beta-catenin (green), a marker of cell borders and cobblestone morphology seen at col-4 conditions of higher concentration (minimum 12 mg/mL) and thickness (minimum 1 mm). All images were taken at 40× magnification.

FIG. 7 provides representative images of corneal endothelial cells (immortalised cell line) cultured on col-4 membranes compared to the standard method of culturing (on coated plastic wells) as shown in the control images. Expansion was monitored over 1 week and images shown represent cells at Day 0 (DO) the first day following seeding, Day 4 (D4) and Day 6 (D6), the final day of observation. Col-4 demonstrated cell expansion and normal morphology when compared to control images. All images were taken at 10× magnification.

FIG. 8 provides representative images of corneal endothelial cells stained with 3 different cell markers, ZO-1, Ki-67 and Na⁺K-ATPase (al) in both the control condition (B, D, F) and when grown on a col-4 membrane (A, C, E). Nuclei (blue) are stained with Hoescht. All images were taken at 20× magnification.

FIG. 9 provides representative images of primary endothelial cells cultured on col-4 membranes compared to a standard culturing method (on col-1 coated wells). Images were taken over the course of a week with representative images shown from Day 0 (DO), Day 3 (D3) and Day 6 (D6). All images were taken at 10× magnification.

FIG. 10 provides a representative image depicting the mechanical strength of a col-4/laminin membrane demonstrated by the ease with which it can be picked up and moved around using tweezers.

### DETAILED DESCRIPTION

The present inventors have developed printable collagen bioinks using type IV collagen with mechanical and structural properties that may facilitate application to tissue in a structured form. The bioinks of the present invention may be compatible with cells, e.g., lens epithelial cells, and/or may support the growth and/or proliferation of corneal endothelial cells. The compositions of the present invention may be used to apply collagen gels to tissue (e.g. eye) using two- or three-dimensional (extrusion) bioprinting techniques. The compositions may provide a means of delivering agents to biological targets (e.g. organs, tissues, cells). While suitable for application to the cornea, the compositions described herein provide a platform for numerous applications in the areas of tissue repair and/or regeneration and the delivery of agents by virtue of providing, for example, structural support, viable cells, and other factors.

There is a need in the art for effective collagen-derived bioinks which may aid tissue repair and/or regeneration by providing replacement tissue and/or replacement cells. The compositions described herein are based on type IV collagen The compositions of the present invention are also ideal agents for the delivery of a diverse range of growth factors and other agents. The compositions described herein may utilise biomaterial that mimics in vivo tissue and acts as a scaffold for cells to populate, and/or, through the manipulation of conditions, encourage the cells themselves to regenerate their surrounding matrix. In the context of their suitability for application to eye tissue the present inventors have, for example, addressed the difficulties of creating a matrix that can embody the structural integrity of the tissue under treatment (e.g. cornea) whilst maintaining transparency and still being porous and biocompatible enough to allow for the infiltration, migration and/or proliferation of corneal cells and growth factors.

The balance between providing the nutritional needs of damaged tissue while meeting the structural, mechanical and physical requirements of damaged tissue (e.g. eye tissue such as cornea) was a problem existing at the time that the present invention arose. The present invention provides improved compositions and methods for delivering agents to a broad variety of biological targets. Without limitation to any particular application, the compositions may be used for tissue repair and/or regeneration.

Compositions for Delivery of Biological Agents

The present invention provides compositions suitable for the delivery of agents to biological targets such as tissues and cells. The compositions may also be used for tissue repair and/or regeneration.

The compositions may utilise a base scaffold material to provide structural support upon application to a biological target (e.g. tissues, membranes, cells, organs), to facilitate the delivery of agents to the biological target.

The scaffolds may be collagen scaffolds. These may be generated, for example, via the use of type IV collagen in the compositions. The type IV collagen used may be unmodified when compared to its naturally occurring counterpart. Modified type IV collagen may also be used. In some embodiments, the modifications are at the end of the collagen fibre.

The compositions of the present invention may further optionally comprise ions and/or one or more sources of ions. Non-limiting examples of suitable ions include calcium ions and sodium ions. Non-limiting examples of suitable ion sources include compounds comprising calcium (e.g. calcium chloride) and sodium (e.g. sodium chloride). Calcium ions and sodium ions may be present in the compositions of the present invention together or individually.

The present inventors have identified optimal relative concentrations of type IV collagen, sodium ions and/or calcium ions for the compositions of the present invention, some of which are described in the Examples and claims of the present application. It will be understood that the relative concentrations of type IV collagen, sodium ions and/or calcium ions disclosed are exemplary only.

The composition may comprise 6-24 mg/ml type IV collagen, 0.04-0.15 M sodium ions and/or 0.008-0.4 M calcium ions. The composition may comprise 1-20 mg/ml type IV collagen, 0.07-0.5 M sodium ions and/or 0.008-0.4 M calcium ions. In some embodiments, the composition may comprise 1-20 mg/ml type IV collagen, 0.06-0.25 M sodium ions and/or 0.008-0.1 M calcium ions. The composition may comprise 3-15 mg/ml type IV collagen, 0.06-0.25 M sodium ions and/or 0.008-0.4 M calcium ions. Other potential ranges for the components of the composition include 3-15 mg/ml type IV collagen, 0.06-0.1 M sodium ions and/or 0.01-0.05 M calcium ions. In some embodiments, the composition may comprise 4-12 mg/ml type IV collagen, 0.06-0.08 M sodium ions and/or 0.015-0.03 M calcium ions. Alternatively, the composition may comprise 5-10 mg/ml type IV collagen, 0.06-0.07 M sodium ions and/or 0.018-0.02 M calcium ions. In some embodiments, the composition may comprise less than 15 mg/ml type IV collagen and more than 0.06 M sodium ions and/or more than 0.018 M calcium ions.

The compositions of the present invention may further comprise one or more crosslinking agents. One non-limiting example of a suitable crosslinking agent is riboflavin. The riboflavin could be present at a concentration of 0.01-0.5% (w/v). The riboflavin could be present at an amount of about 0.01-0.1 mg. In some embodiments, the amount of riboflavin present is 0.01, 0.1 mg or any amount between these values. Light, such as UV light or blue light could be used to activate the riboflavin, crosslinking the composition. The person skilled in the art will be aware of other suitable crosslinking agents and/or light sources, for example, Rose Bengal dye and green light, both of which have been approved for several applications to the cornea. In some embodiments, Rose Bengal is used as a photocrosslinking agent and is activated by green light. Alternatively, Rose Bengal is used as a photocrosslinking agent and is activated by white light. In some embodiments, 0.01-0.5% (w/v) Rose Bengal is used for photocrosslinking. In further embodiments, compositions provided by crosslinking the collagen bioink with Rose Bengal and a suitable light source will be coloured. In some embodiments, the colour will be pink. These coloured compositions may be used for monitoring collagen metabolism in tissue, or for other uses requiring tracking of collagen activity. In other embodiments, crosslinking agents may comprise fibrinogen and/or thrombin.

The compositions may further comprise cells. The cells may, for example, be mammalian cells (e.g. human cells, canine cells, feline cells, bovine cells, porcine cells, equine cells, caprine cells, hircine cells, murine cells, leporine cells, cricetine cells, musteline cells, or any combination thereof). The type of cells utilised will generally depend on the specific purpose for which the composition is to be used. For example, the cells may be of the same type as a tissue to which the composition is to be administered (e.g. eye surface cells including those of the central and/or peripheral corneal epithelium, bulbar and/or tarsal conjunctival epithelia, tarsal conjunctival stroma, and/or lid margin; skin cells including but not limited to keratinocytes, melanocytes, Merkel cells, and Langerhans cells; and neural tissue cells including but not limited to neurons and glial cells). Other examples include epithelial cells, keratocytes, neuronal cells, photoreceptor cells, müller cells and endothelial cells. The endothelial cells may be primary endothelial cells. In some embodiments, the cells may be hematopoietic stem cells, bone marrow stem cells, neural stem cells, epithelial stem cells, skin stem cells, muscle stem cells, adipose stem cells, pluripotent stem cells, induced pluripotent stem cells, embryonic stem cells, mesenchymal stem cells, or any combination thereof. In some embodiments, the cells may be neuronal cells. One non-limiting example of suitable epithelial cells is lens epithelial cells. A non-limiting example of suitable endothelial cells is corneal endothelial cells. The cells of the compositions may be autologous (i.e. self-derived from a given subject intended to receive the composition, or allogeneic (i.e. donor-derived).

The compositions of the present invention may comprise essential and/or non-essential amino acids. Non-limiting examples of suitable essential amino acids include isoleucine, leucine, lysine, methionine, phenylalanine, threonine, tryptophan, valine, cysteine, tyrosine, histidine and arginine.

The compositions of the present invention may comprise additional components (e.g. agent/s) including, but not limited to, fibronectin, anaesthetics, antibiotics, hormones (e.g. insulin), growth factors (e.g. human epidermal growth factor (hEGF), platelet derived growth factor, vascular endothelial growth factor, fibroblast growth factor (FGF), epithelial growth factor, transforming growth factor [including beta], and connective tissue growth factor), fibrin stabilizing factors (e.g. factor XIII), matrix protein/s (e.g. collagen [such as type I collagen], laminin, integrin), vitamins (e.g. vitamin C, riboflavin), glycoproteins (e.g. transferrin), Fetal Bovine Serum (FBS), Fetal Calf Serum (FCS), human serum, platelet lysate, human platelet lysate, therapeutic drugs and any combination thereof. In some embodiments, the composition comprises a culture medium comprising the ions and amino acids. Non-limiting examples of suitable growth factors include vascular endothelial growth factor (VEGF) and fibroblast growth factor (FGF). The vitamins may be ascorbate (vitamin C) and/or riboflavin. The matrix proteins could include, but are not limited to, type I collagen; and/or laminin.

The compositions of the present invention may include other suitable ingredients including water and/or culture medium (e.g. DMEM, DMEM/F-12, MEM, CnT-PR). The culture medium may comprise, for example, any one or more of Glycine, L-Alanine, L-Arginine hydrochloride, L-Asparagine-H2O, L-Aspartic acid, L-Cysteine hydrochloride-H2O, L-Cystine 2HCl, L-Glutamic Acid, L-Glutamine, L-Histidine hydrochloride-H2O, L-Isoleucine, L-Leucine, L-Lysine hydrochloride, L-Methionine, L-Phenylalanine, L-Proline, L-Serine, L-Threonine, L-Tryptophan, L-Tyrosine disodium salt dihydrate, L-Valine, Vitamins, Biotin, Choline chloride, D-Calcium pantothenate, Folic Acid, Niacinamide Pyridoxine hydrochloride, Riboflavin, Thiamine hydrochloride, Vitamin B12, i-Inositol, Inorganic Salts, Calcium Chloride (CaCl2)) (anhyd.), Cupric sulfate (CuSO4-5H2O), Ferric Nitrate (Fe(NO3)3″9H2O), Ferric sulfate (FeSO4-7H2O), Magnesium Chloride (anhydrous), Magnesium Sulfate (MgSO4) (anhyd.), Potassium Chloride (KCl), Sodium Bicarbonate (NaHCO₃), Sodium Chloride (NaCl), Sodium Phosphate dibasic (Na2HPO4) anhydrous, Sodium Phosphate monobasic (NaH2PO4-, Zinc sulfate (ZnSO4-7H2O), Other Components, D-Glucose (Dextrose), Hypoxanthine Na, Linoleic Acid, Lipoic Acid, Putrescine 2HCl, Sodium Pyruvate, Thymidine, or any combination thereof.

In some embodiments, ions are provided as components of an ionic salt included in the composition.

Non-limiting properties of the compositions include one or more of the following:
Non-Newtonian shear-thinning fluid properties, whereby the viscosity of the composition may decrease as the shear-rate increases. In some embodiments, the viscosity of the compositions may be in the range of 0.01 and 1000 Pa·s at room temperature.
Optical clarity without impeding or without substantially impeding vision arising from transmittance of light, for example, over 90% in the visual colour range of 400-700 nm.
Suitability for 2D and/or 3D printing (e.g. bioprinting/extrusion printing) with capacity to maintain or substantially maintain shape/structure following printing.
Suitability for printing while maintaining the viability of cells within the composition during the printing process.
Capacity to be provided in two- or three-dimensional structure with or without the inclusion of viable cells.
Capacity to sustain and/or promote the growth of cells (e.g. sustain and/or promote the expansion growth of primary human cells such as epithelial cells (e.g. lens epithelial cells), keratocytes, neuronal cells, and endothelial cells (e.g. corneal endothelial cells).
Capacity to promote the formation of spheroid organoids.
Capacity for degradation by cells over time (e.g. 2-7 days).
Maintenance of cell viability over time (e.g. 7 days at 34° C.).
Capacity to adhere to various surfaces, including tissues, organs, membranes (e.g. mammalian and human tissues, organs, membranes).

### Preparation of Compositions

In general, the compositions of the present invention may be prepared by combining a plurality of different preparations. Lyophilised bovine type IV collagen may be used in the preparation of the compositions. Additionally or alternatively, human collagen may be used. The type IV collagen may be neutralised prior to the addition of ions and other components of the compositions. A person skilled in the art would recognise that various buffer solutions could be used to keep the collagen at physiological pH and to maintain solubility.

In some embodiments, the invention provides devices and/or kits for the preparation of the compositions. The devices and/or kits may facilitate the separation of different preparations needed to form the compositions of the invention until use.

The devices and kits may further comprise a component providing a means to facilitate mixing of the two compartmentalised preparations such as, for example, by removal of barrier/s separating the first and second compartments, and/or by puncturing a seal or wall of either or both compartments. The skilled person will readily understand that various arrangements can be made for this purpose.

Additionally or alternatively, devices and kits may be configured in a manner that ensures mixing of the two compartmentalised preparations during or following release of the preparations from the device or kit.

In some embodiments, the devices and kits may comprise additional compartments comprising additional components to be used in generating the composition (e.g. platelet lysate, ions, amino acids, cells, antibiotics, growth factors, fibrin stabilizing factors, anaesthetics and so on). The device or kit may be configured in such a way to facilitate mixing of these additional components with each other and/or with the other components of the compositions.

The devices and kits may facilitate mixing of separated components prior to, during or immediately following discharge of the components from the device or kit.

In some embodiments, the compositions are bioinks and the device is a three-dimensional (3D) printer (e.g. an extrusion printer).

In some embodiments of the present invention, the crosslinking agent is riboflavin. In further embodiments, the riboflavin may be activated by UV light or blue light. The solution, which may contain type IV collagen and sodium and/or calcium ions, may be extruded in a line and UV light or blue light may be applied. Additional lines may be applied on top of the first line to form a structure which will be crosslinked by the crosslinking agent and the application of light.

In some embodiments, crosslinking occurs in under 15 minutes, under 14 minutes, under 13 minutes, under 12 minutes, under 11 minutes, under 10 minutes, under 9 minutes, under 8 minutes, under 7 minutes, under 6 minutes, under 5 minutes, under 4 minutes, under 3 minutes, under 2 minutes or under 1 minute. The light source used may be 3 mW/cm² 365 nm UV, 10 mW/cm² blue light or a tissue culture hood UV lamp.

The crosslinking agent may be Rose Bengal. In some embodiments, Rose Bengal is activated by green light. In further embodiments, Rose Bengal is activated by white light. In still further embodiments, crosslinking occurs in under 15 minutes, under 14 minutes, under 13 minutes, under 12 minutes, under 11 minutes, under 10 minutes, under 9 minutes, under 8 minutes, under 7 minutes, under 6 minutes, under 5 minutes, under 4 minutes, under 3 minutes, under 2 minutes or under 1 minute. The light source used may be 100 mw/cm² white light. The person skilled in the art would realise that the light source and parameters could be varied according to the particular application.

The collagen gels of the present invention may have various thicknesses according to the application, for example, the thickness of a gel could be between 50 μm to 3 mm.

### Applications of the Compositions

The present inventors have developed compositions for the delivery of agent/s to target tissues and cells and for the repair and/or regeneration of tissue with characteristics making them highly suitable for bioprinting. The compositions of the present invention may be used in applications where there is a need for delivery of agents (e.g. natural growth factors, drugs, nanoparticles, and/or cells,) and/or for the fixation of individual biological surfaces, and/or in tissue culture methods.

In some embodiments the compositions can act as tissue sealants and/or as a fixative for biological structures. They may provide structural and/or nutritional support to tissue. Additionally or alternatively, the compositions may facilitate the growth of target cell type/s, including those which may be provided as a component of the compositions and/or cells present in the target tissue. The compositions could be useful for culturing cells.

In some embodiments, compositions provided by crosslinking the collagen bioink with Rose Bengal and a suitable light source will be coloured. In some embodiments, the collagen compositions may be pink. These coloured compositions may be used for monitoring collagen metabolism in tissue, or for other uses requiring tracking of collagen activity.

While no limitation exists as to the type of tissue to which the compositions may be applied, the present inventors have demonstrated that the compositions are compatible with cells, e.g., lens epithelial cells and/or may support the growth and/or proliferation of corneal endothelial cells.

For example, the compositions are demonstrated herein to be effective in supporting the growth and/or proliferation of corneal endothelial cells. In these embodiments, the compositions may be used to promote the proliferation and/or migration of corneal endothelial cells. The compositions may, for example, support multidirectional growth and/or stratification of corneal epithelial cells, which may partially or completely biodegrade the composition once a cell monolayer is formed.

The present invention thus provides methods for the delivery of a variety of agents to biological targets. The targets may, for example, be located in or around eye tissue including tissue of the central and/or peripheral corneal epithelium, bulbar and/or tarsal conjunctival epithelia, tarsal conjunctival stroma, Descemet's membrane and/or lid margin.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific Examples, which should not be construed as in any way limiting.

### Example One: Preparation and Characterisation of a Soluble Type IV Collagen (Col-4) Solution Capable of Crosslinking at Room Temperature (RT)

Col-4 powder (minimum concentration of 6 mg/mL) was dissolved in 0.1M acetic acid, then neutralised with 5M NaOH and 27.4 mg/mL CaCl₂) to a final pH of between 6.7 and 7.4. These steps were conducted at RT. Once the solution was neutralised, 0.1-0.2 mg of riboflavin was added for every 90 μL of col-4 originally used and dissolved by further vortexing and centrifuging the col-4/riboflavin solution for 30 seconds and 1 minute, respectively. A range of col-4 concentrations were tested from 6 mg/mL to 24 mg/mL.

Compared to the use of type I collagen (col-1) the col-4 method uses just over half the 5M NaOH required, as shown in Table 1.

TABLE 1
A comparison of quantities required for col-1 and col-4
photocrosslinkable solutions using 12 mg/mL collagen.

| Quantities for COL-I | Quantities for COL-4 |
|---|---|
| 90 μL of 12 mg/mL COL-I | 90 μL of 12 mg/mL COL-IV |
| solution dissolved in 0.1M | solution dissolved in 0.1M |
| acetic acid | acetic acid |
| 3.1 μL of 5M NaOH | 1.8 μL of 5M NaOH |
| 7.3 μL of 27.4 mg/mL CaCl₂ | 7.3 μL of 27.4 mg/mL CaCl₂ |
| 0.1 mg of riboflavin | 0.1 mg of riboflavin |

With the addition of calcium ions, a clear and neutralised col-4 solution was generated. The resulting liquid could be photocrosslinked immediately or stored at −30° C. for use a minimum of 1 week later. The solution could be kept at RT for use a minimum of 1 hour later. Neutralised col-4 without riboflavin could be stored at 30° C. for use a minimum of 1 week later with no change in crosslinking properties. A range of col-4 solutions were tested from 6 mg/mL to 24 mg/mL. Col-4 solution at 6-18 mg/mL after photocrosslinking and rinsing remained as transparent structures. However, col-4 solution at 24 mg/mL after crosslinking and rinsing, dissolved into pieces and could not hold its 3D structure.

### Photocrosslinking and Rheological Tests

In the rheological tests, 100 μL of col-4 solution was pipetted centrally onto the bottom plate of the rheometer. A parallel plate was used to condense and spread the droplet to completely cover the bottom plate area. The rheometer was allowed to stabilise for 3 minutes. The liquid was exposed to either UVA or blue light (400-500 nm) for 3 minutes. The gelation point was determined when G′>G″, indicating that the solution had become more viscous than elastic.

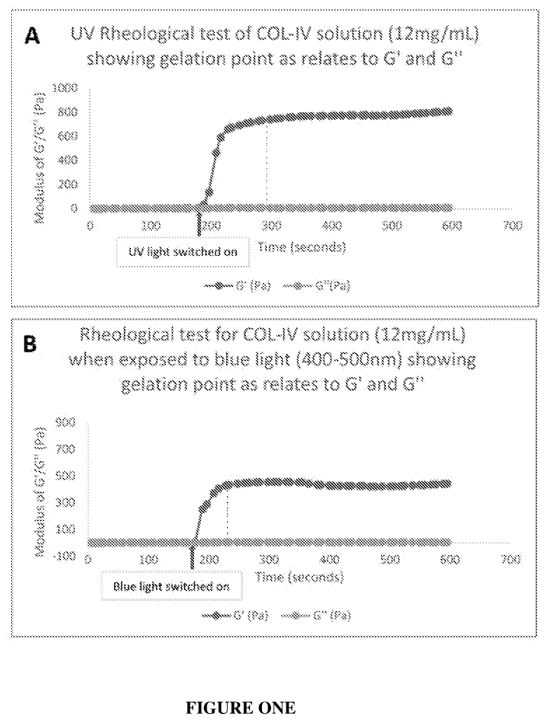

Rheological tests demonstrated that initially G′ (measure of viscosity) was lower than G″ (measure of elasticity) indicating the solution was initially more liquid/watery. Filters for 365 nm (UV wavelength) and 400-500 nm (blue light) were used. The gelation starting point under 365 nm (UV) occurred within 10.24 seconds of the solution being exposed to the light; and the full crosslinking time was about 3 min (FIG. 1A). The gelation starting point under 400-500 nm (blue light) occurred almost immediately indicating a faster crosslinking time under blue light as full crosslinking occurred after 1 min of exposure (FIG. 1B). Full crosslinking times are shown by dotted lines in FIGS. 1A and 1B. Both the UV light and blue light used were at a power of 3 mW/cm². The results indicate a faster crosslinking time under blue light, however, the average G′ achieved under blue light was 442 Pa, which was weaker than achieved under UV at 810 Pa (FIG. 1).

### Printability Tests

To evaluate the printability of col-4, a pore size test was conducted using an Edu3D printer at the University of Wollongong TRICEP facility. The col-4 solution was extruded through a syringe tip and printed as a 9×9 mm 2-layered lattice under exposure to blue light (wavelength of 405 nm). Multiple parameters including temperature, flow rate of the ink, and speed of printing were adjusted to determine the optimum settings to print the col-4 as a bioink. Printability was determined by calculating the internal perimeter (L) and area (A) of 6 pores visible in the lattice. The average values for L and A were then put into the following formula: Pr=L²/16A. The closer the value was to 1, the more printable the bioink was determined to be.

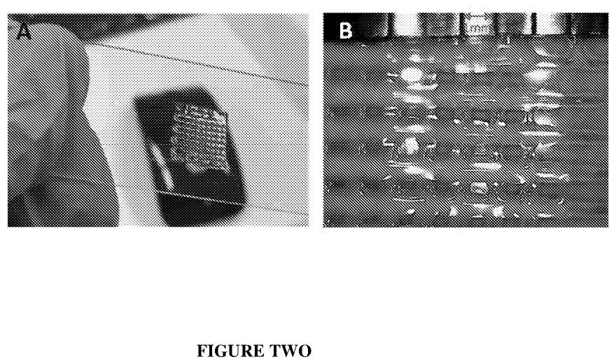

Different parameters were tested on the Edu3D printer including temperature, speed, flow rate and tip diameter. The optimum parameters were determined to be a temperature of 22° C. (RT), a flow rate of 0.8 mm/min (indicating the amount of bioink extruded), a printing speed of 150 mm/min and extrusion through a 25GA tip of diameter 0.26 mm. The printability under these conditions was determined to be 0.99. This value was very close to 1 indicating the structure held its shape well and can be designated as printable (FIG. 2A/B).

Generation of Col-4 Scaffolds with Varying Thicknesses

The ability to crosslink allowed for the generation of col-4 scaffolds with varying thicknesses. Varying amounts of solution were used to create different shapes/moulds.

### Generating a Collagen Gel

To generate a gel of 5 mm thickness, a col-4 solution was created as described above. A mould was created by cutting the tip off a 1 mL syringe (with a diameter of 5 mm). The plunger within the syringe was also cut to be flat and covered in parafilm. The modified syringe was held upright by blu-tack to stand directly under UV or blue light. The plunger of the syringe was set 5 mm below the top to allow the solution added to crosslink into a gel of 5 mm thickness. 90 μL of col-4 solution was required to fill the space in the syringe. The col-4 solution in the syringe was exposed to UV/blue light for 10 minutes to ensure crosslinking through the entire thickness. This method with syringes of varying diameter can be used to create gels of different thicknesses and dimensions.

### Generating a Collagen Membrane

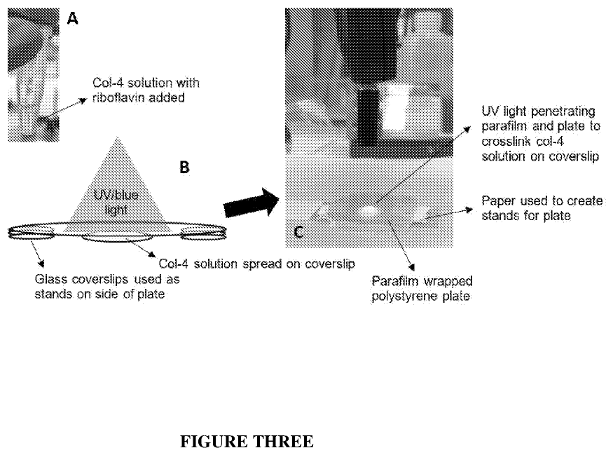

To generate a col-4 membrane of approximately 50 μm thickness and 13 mm diameter, 25 μL of col-4 solution (FIG. 3A) was pipetted onto a 13 mm circular glass coverslip. The glass coverslip served as a supporting base for the col-4 solution. Col-4 could also be crosslinked on plastic such as polystyrene or parafilm, which is a mixture of waxes and polyolefins. When a glass coverslip was used as a supporting base, as shown in FIG. 3B/C, a parafilm-wrapped transparent polystyrene plate was then placed on top of the droplet. The solution was spread to fill the area and photocrosslinked using UVA light (3 mW/cm² power, 3 cm above the coverslip sample). Following crosslinking, the col-4 membrane was washed 2×15 min in PBS until transparent. By adjusting the height of stands and the area of the supporting base (e.g. the glass coverslip or paper), the thickness and diameter of the membrane could be easily adjusted.

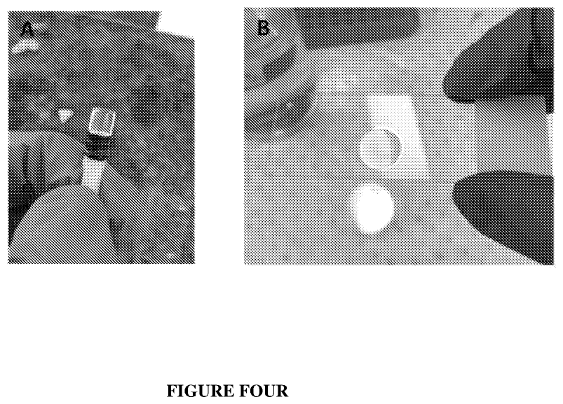

The methods above allowed for the creation of thin membranes which may be used in culturing experiments to create corneal endothelial sheets and thick gels. The moulding method to create a collagen gel described above could be used to generate a thick col-4 scaffold, for example a 5 mm thick col-4 cylindrical scaffold could be generated (FIG. 4A). Using the method described above for generating a membrane, various membrane thicknesses could be generated, one example was a 50 μm membrane (FIG. 4B) The resulting membrane structures were attached to the base supporting materials, for example a glass coverslip (FIG. 4B). These results showed that col-4 photocrosslinkable solutions can be used to generate structures of all shapes and forms.

### Transparency Testing of the Col-4 Membrane

The ColorQuest XE at the AIIM facility, University of Wollongong, was used for transparency testing. The machine was standardised by measuring light transmittance through a black plate. Col-4 membranes (of approximately 50 μm thickness) adhered to transparent coverslips where loaded into the machine and total light transmittance was measured.

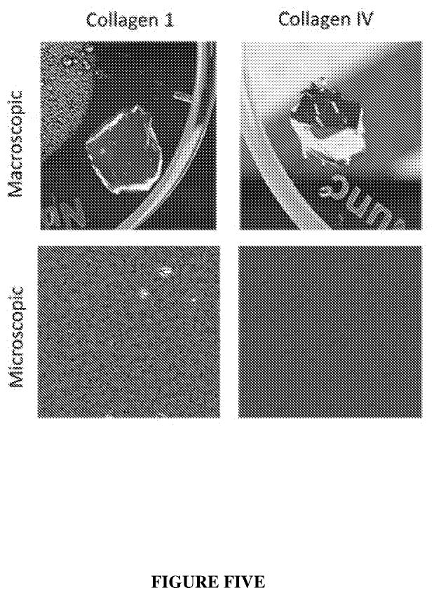

The average light transmittance of 3 samples was 90.4% which matches natural corneal light transmittance. One sample detached from the coverslip was found to have a light transmittance of 91.13%, demonstrating that the coverslip did not significantly alter the light transmittance of the material. In comparison, 3 samples of col-1 prepared and tested in the same way had an average light transmittance of 86.2%. These transparency differences can also be seen macroscopically and microscopically when comparing the col-1 and col-4 biomaterials. (FIG. 5)

### Cell Compatibility

Both lens cells and corneal endothelial cells were used to evaluate the cell compatibility of the col-4 solution by culturing the cells on top of col-4 membranes.

### Lens Epithelial Cells on a Col-4 Membrane

Lens epithelial cells were obtained by collecting the primary lens epithelium (attached to its native membrane, the lens capsule) from postnatal rats as explants. In this procedure, the eye was removed from euthanised rats, torn open posteriorly via the optic nerve and the lens was removed. The posterior side of the lens was determined and the lens capsule was torn and peeled to the lens equator. The anterior side of the lens capusle containing the lens epithelium was visible as a thicker piece of tissue and was isolated as the lens fibre cell mass was removed and discarded. This lens epithelial sheet was then pinned down to the dish (to form the explant) by applying pressure at the edges using tweezers, and cultured in M99 media (M199 concentrate with Earle's salts, L-glutamine & sodium bicarbonate, adjusted to pH 7.2, supplemented with Amphotericin B (250 μg/mL), bovine serum albumin (1 mg/mL), Pen/Strep (10,000 U/mL penicillin & 10,000 μg/mL streptomycin) & L-glutamine (68.4 mM). Both thin films and thick gels of col-4 were created to test for differing effects of collagen quantity on the lens cells. 50 μm thin films and 1 mm thick gels were created as described above. The lens epithelium was exposed to col-4 by pinning these collagen films/gels onto the top of the explant so the epithelial cells were directly exposed to the collagen. The control condition was defined as the condition where lens epithelial explants were not exposed to any collagen.

Col-1 thin films and thick gels were also generated and used as a contrast group. Lens epithelial cells were exposed to col-1 in the same way as they were exposed to col-4, as described above.

After one week of observation, the collagen was removed and the lens explants were fixed with 10% neutral buffered formalin (NBF) (10 minutes) and rinsed (3×5 min) and stored in 70% ethanol until immunostaining. Lens epithelial explants were stained with beta-catenin (epithelial cell membrane marker) and α-SMA (mesenchymal cell marker). This immunostaining was done differently compared to corneal endothelial sheet staining. Explants were hydrated with 3×5 min washes in PBS/BSA at RT and permeabilised (3×5 min) in PBS/BSA/Tween-20. Explants were rinsed 2×5 min in PBS/BSA. Excess buffer was removed to create a thin film of liquid over the explants. 60μL of 3% normal goat serum (NGS) was applied to the explants and left for 30 minutes at RT for blocking. Primary antibodies diluted in 3% NGS were applied to the explants. Explants were incubated overnight at 4° C. in humidified chamber. Explants were rinsed 3×5 min in PBS/BSA. Secondary antibodies were diluted in PBS/BSA and added to explants. Explants were incubated in a dark, humidified chamber for 2 hours. Explants were rinsed 3×5 min in PBS/BSA before the addition of 1 mL of Hoechst (diluted in PBS/BSA) into the dishes for 5 minutes. Final 2×5 min PBS/BSA washes were done before mounting explants with coverslips with 10% PBS/glycerol.

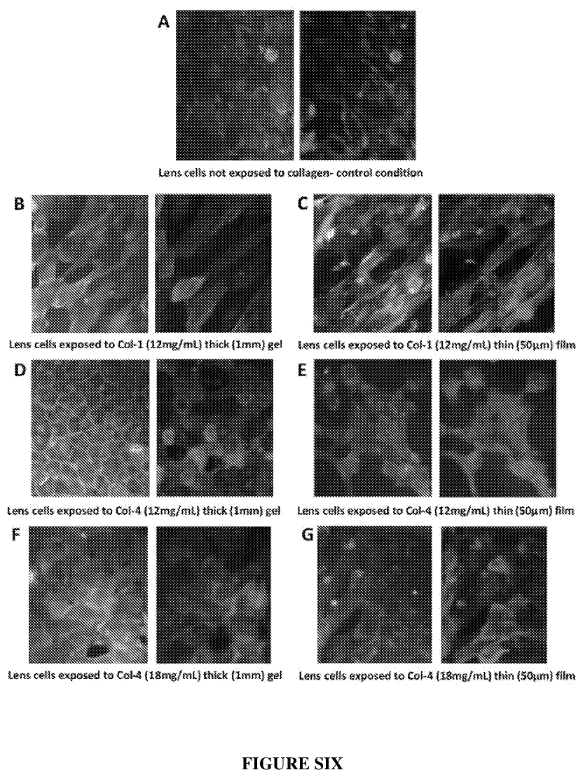

There were significant differences in the biocompatibility of lens epithelial cells when exposed to col-4 compared to col-1. Lens epithelial cells exposed to col-1 transformed into myofibroblastic cells. These myofibroblastic cells were elongated cells and when stained with α-SMA they showed distinct actin stress fibres, a marker of mesenchymal cells (typical of pathological cells found in cataracts). In comparison, cells exposed to col-4 (the normal constituent of the lens capsule) that were stained with beta-catenin, a marker of lens epithelial cell integrity, showed strong membrane staining around epithelial cell borders (FIG. 6). This staining was especially prominent in conditions where the cells were exposed to greater concentrations of col-4; the 12 mg/mL and 18 mg/mL conditions. Hoechst was used to counter-label nuclei (blue; FIG. 6) The effect was also greater with exposure to greater quantities of col-4 i.e. the conditions where thick (1 mm) gels were applied.

To conclude, a minimum concentration of 12 mg/mL of col-4 was required to maintain lens epithelial cells while this same concentration of col-1 leads lens epithelial cells to transform into myofibroblastic (cataractous) cells.

### Culturing Corneal Endothelial Cells on a Col-4 Membrane

Using the method described above for generating a collagen membrane, a clear 50 μm thick of col-4 membrane was generated. An immortalised corneal endothelial cell line (B4G12) was cultured on the col-4 membrane (at a cell density of 1×10⁵ cells) in 5% FCS media (1:1 mixture of Nutrient Mixture Ham's F12 and Medium 199, 5% FCS, 20 μg/mL ascorbic acid, 10 ng/mL FGF-2 & 10,000 U/mL penicillin and 10,000 μg/mL streptomycin) and 5% hPL media (same components but 5% FCS was substituted for 5% hPL).

Primary endothelial cells received from donor corneal rims were also cultured on col-4 membranes. The remaining Descemet's membrane was dissected away from the corneal rim and pieces were placed in 500 μL of collagenase A (2 mg/mL) for 4 hours to be digested. Following digestion, the tube was centrifuged at 190 g for 5 min at 20° C. Collagenase was removed and replaced with 100 μL of TrypLE (trypsin solution). The sample tube was placed in a 37° C. water bath for 10 minutes and agitated halfway through. 400μL of M5 (maintenance media—human endothelial SFM, 5% FCS and 1% Pen/Strep) media was added to deactivate the TrypLE. The solution was centrifuged as before. The supernatant was removed without disturbing the cell pellet, then 75 μL of M5 media was added. The cells were resuspended and pipetted onto the col-4 membrane (previously placed in a 12 well plate) and left to adhere for 45 mins at 37° C. Following cell adhesion, 500μL of M5 media was added into the well. The following day, M5 media was replaced with M4 media (proliferation media—same mixture as 5% FCS media).

In comparisons, the standard method was used to culture cells which involved coating plastic wells of a 12-well plate with a col-1 solution (col-1 solution diluted 1:20 in 20 mM acetic acid). After digestion, cells were then seeded onto these coated wells in control conditions.

Once corneal endothelial cells (both cell line and primary cells) reached full confluence, the cells were immunostained for corneal endothelial cell markers: zonula occuldins-1 (ZO-1) and Na⁺K-ATPase. Laminin (01), an extracellular matrix protein secreted by corneal endothelial cells and the cell proliferation marker, Ki-67, were also stained to examine the activity of cells. Immunostaining was done by initially fixing the sheets in 4% paraformaldehyde. Following fixing, the sheets were stored in 1×PBS until staining. When staining, 0.5% Triton X/PBS was first used to incubate the sheets for 15 minutes at RT. The sheets were then incubated in 5% BSA/PBS for 30 minutes at RT for blocking. Primary antibodies were applied and left overnight to incubate at 4° C. Primary antibodies (diluted in 1% BSA/PBS) were removed and sheets were washed 3×5 min in 1×PBS. Secondary antibody (diluted in 1% BSA/PBS) and Hoechst (diluted 1:1000) was added to the sheets and left to incubate at RT for 2 hours in a dark humidified chamber. Sheets were washed 3×5 min in 1×PBS and mounted using 20% PBS/glycerol on coverslips.

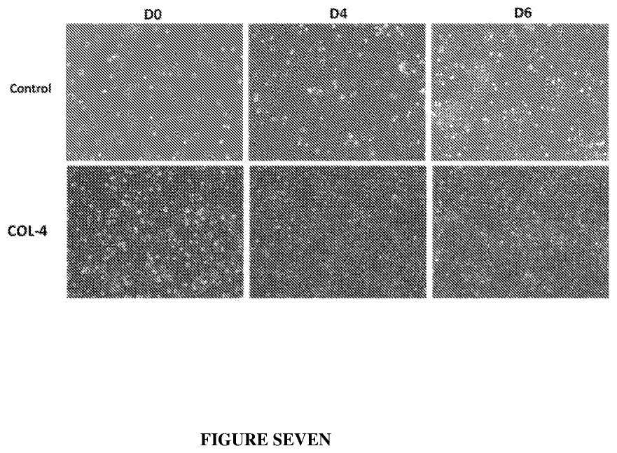

Corneal endothelial cell line cultured on col-4 membranes achieved an average density of 3784 cells/mm² compared to cells cultured in the standard way on a coated plastic well surface at an average density of 2282 cells/mm² (FIG. 7). The reason for the difference in cell density between col-4 and controls was the difference in area cells were provided to expand on. In the control condition 1×10⁵ cells expanded on the well of a 12-well plate which has a significantly greater area compared to the 13 mm col-4 membranes. The cells on col-4 membranes also demonstrate a clear hexagonal shape characteristic of corneal endothelial cells (FIG. 7).

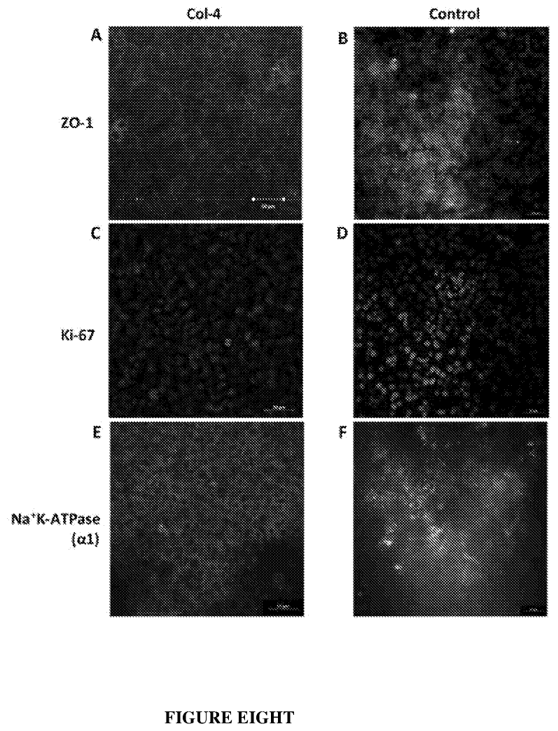

Immunostaining results showed that the cells on col-4 expressed strong endothelial markers ZO-1 (cell border marker of tight junctions) and Na+K-ATPase (marker of sodium-potassium pumps) (FIG. 8). A strong staining with Ki-67 was detected which indicated cell proliferation (FIG. 8).

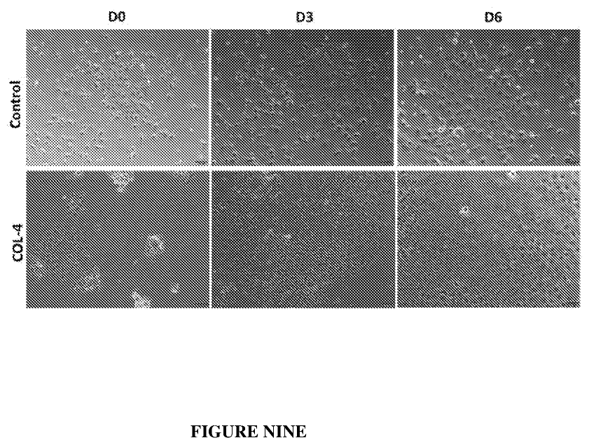

Primary endothelial cells also proliferated well on col-4 membranes in comparison to standard culturing methods where cells were grown on col-1 coated wells (control), as shown in FIG. 9. Cells grown on col-4 appeared to expand into better patches of endothelial cells with hexagonal morphology in comparison to the fibroblastic-like cells in the control group which retained a more stem-like character. Therefore, col-4 membranes appear to facilitate the growth of primary endothelial cells from patients which have limited proliferative ability.

### Col-4 Solution as a Carrier for Other Molecules

The col-4 bioink can be modified by the addition of other molecules. Laminin (another extracellular matrix protein) was used as an example. The laminin added was from murine sarcoma basement membrane and is also used as a component of coating solution used in culturing wells to grow corneal endothelial cells. 10μL of laminin (stock: 10 μg/mL) was added to 90 μL of col-4 solution (12 mg/mL), so that the final concentration of laminin was 1 μg/mL. Within a normal coating solution, laminin is approximately 1 μg/mL within a solution of chondroitin sulphate. The solution was crosslinked as described above. Following the addition of riboflavin, 90 μL of col-4 solution was transferred to a new eppendorf tube. 10 μL of laminin previously thawed (originally stored at −20° C.) was added to the col-4 solution. The new solution was vortexed for 20 seconds and centrifuged for 5 seconds. Collagen-laminin membranes were created using the method described above. Transparent membranes created following a wash were stored at 4° C. until use.

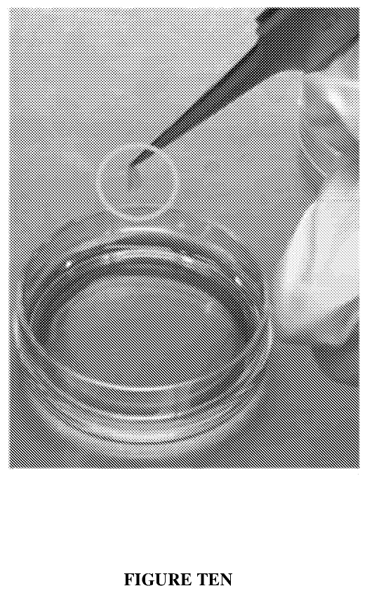

With the incorporation of laminin the col-4 solution remained crosslinkable and resulting membranes retained normal transparency as observed by the human eye. Similar mechanical strength was also observed for the col-4/laminin membranes compared to col-4 only membranes (FIG. 10). This strength was observable through the easy handling of the membrane with tweezers which was a feature also seen for the col-4 only membranes. This showed that the col-4 ink developed has the potential to carry other molecules and still remain crosslinkable.

1. A composition comprising:
6-24 mg/ml type IV collagen;
0.04-0.15 M sodium ions and/or 0.008-0.4 M calcium ions; and
one or more crosslinking agents.

2. The composition of claim 1, wherein the composition comprises 0.01-0.1 mg riboflavin.

3. The composition of claim 1 or claim 2, wherein the one or more crosslinking agents are capable of activation by light.

4. The composition of claim 3, wherein the light is UV light, blue light, green light or white light.

5. The composition of claim 1, wherein the composition comprises fibrinogen and/or thrombin.

6. The composition of any one of claims 1 to 5, wherein the composition further comprises mammalian cells.

7. The composition of claim 6, wherein the mammalian cells comprise or consist of human cells.

8. The composition of claim 6 or claim 7, wherein the mammalian cells comprise any one or more of: neuronal cells, epithelial cells, photoreceptor cells, müller cells, endothelial cells.

9. The composition of any one of claims 6 to 8, wherein the mammalian cells comprise or consist of epithelial cells and/or endothelial cells.

10. The composition of claim 8 or claim 9, wherein the epithelial cells are lens epithelial cells.

11. The composition of claim 8 or claim 9, wherein the endothelial cells are corneal endothelial cells.

12. The composition of any one of claims 1 to 11, further comprising any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.

13. The composition of claim 12, wherein the composition comprises a culture medium comprising the ions and amino acids.

14. The composition of claim 12 or claim 13, wherein:
(i) the growth factors comprise vascular endothelial growth factor (VEGF) and/or fibroblast growth factor (FGF); and/or
(ii) the vitamins comprise riboflavin; and/or
(iii) the matrix proteins comprise type I collagen; and/or
(iv) the matrix proteins comprise laminin.

15. The composition of any one of claims 1 to 14, wherein the ions are components of an ionic salt included in the composition.

16. The composition of any one of claims 1 to 15, wherein the type IV collagen is neutralised.

17. The composition of any one of claims 1 to 16, wherein the composition comprises:
(i) 6-24 mg/ml type IV collagen, 0.06-0.1 M sodium ions, and 0.01-0.05 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

18. The composition of any one of claims 1 to 17, wherein the composition comprises:
(i) less than 24 mg/ml type IV collagen;
(ii) more than 0.04 M sodium ions; and
(iii) more than 0.008 M calcium ions.

19. A method of preparing a composition, the method comprising:
(i) providing a solution comprising:
6-24 mg/ml type IV collagen;
one or more crosslinking agents; and
0.04-0.15 M sodium ions; and/or 0.008-0.4 M calcium ions;
(ii) applying the solution to a surface; and
(iii) activating the one or more crosslinking agents.

20. The method of claim 19, wherein applying the solution to a surface forms a layer, and wherein steps (ii) and (iii) are repeated a plurality of times, wherein each layer is applied on top of the preceding layer.

21. The method of claim 19 or claim 20, wherein the one or more crosslinking agents comprise 0.01-0.1 mg riboflavin.

22. The method of any one of claims 19 to 21, wherein the activating in step (iii) comprises applying light capable of activating the one or more crosslinking agents.

23. The method of claim 22, wherein the light comprises UV light, blue light, green light or white light.

24. A method of preparing a composition, the method comprising:
(i) providing a solution comprising:
6-24 mg/ml type IV collagen;
one or more crosslinking agents; and
0.04-0.15 M sodium ions; and/or 0.008-0.4 M calcium ions; and
(ii) applying the solution to a surface, wherein the solution is divided into at least two components prior to applying to the surface.

25. The method of claim 24, further comprising the steps of:
(iii) adding fibrinogen to at least one component to form formulation (a);
(iv) adding thrombin to at least one component to form formulation (b); and
(v) combining formulations (a) and (b) to form a gel.

26. The method of any one of claims 19 to 25, further comprising adding mammalian cells to the solution and/or the composition.

27. The method of claim 26, wherein the mammalian cells comprise or consist of human cells.

28. The method of claim 26 or claim 27, wherein the mammalian cells comprise any one or more of: neuronal cells, epithelial cells, photoreceptor cells, müller cells, endothelial cells.

29. The method of any one of claims 26 to 28, wherein the mammalian cells comprise or consist of epithelial cells and/or endothelial cells.

30. The method of claim 28 or claim 29, wherein the epithelial cells are lens epithelial cells.

31. The method of claim 28 or claim 29, wherein the endothelial cells are corneal endothelial cells.

32. The method of any one of claims 19 to 31, wherein the solution further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.

33. The method of claim 32, wherein the solution comprises a culture medium comprising the ions and amino acids.

34. The method of claim 32 or claim 33, wherein:
(i) the growth factors comprise vascular endothelial growth factor (VEGF) and/or fibroblast growth factor (FGF); and/or
(ii) the vitamins comprise riboflavin; and/or
(iii) the matrix proteins comprise type I collagen; and/or
(iv) the matrix proteins comprise laminin.

35. The method of any one of claims 19 to 34, wherein the ions are components of an ionic salt included in the mixture.

36. The method of any one of claims 19 to 35, wherein the type IV collagen is neutralised.

37. A composition obtained or obtainable by the method of any one of claims 19 to 36.

38. A method of sealing the surface of tissue, the method comprising applying the composition of any one of claims 1 to 18 or claim 37 to the tissue.

39. A method of delivering agents to tissue, the method comprising applying the composition of any one of claims 1 to 18 or claim 37 to the tissue.

40. A method of culturing cells, the method comprising applying the cells to the composition of any one of claims 1 to 18 or claim 37.

41. The method of claim 40, wherein the cells comprise or consist of epithelial cells and/or endothelial cells.

42. The method of claim 41, wherein the epithelial cells are lens epithelial cells.

43. The method of claim 41, wherein the endothelial cells are corneal endothelial cells.

44. A composition of any one of claims 1 to 18 or claim 37 for use in sealing the surface of tissue.

45. A composition of any one of claims 1 to 18 or claim 37 for use in delivering agents to tissue.

46. A composition of any one of claims 1 to 18 or claim 37 for use in culturing cells.

47. The use of claim 46, wherein the cells comprise or consist of epithelial cells and/or endothelial cells.

48. The use of claim 47, wherein the epithelial cells are lens epithelial cells.

49. The use of claim 47, wherein the endothelial cells are corneal endothelial cells.

50. A kit, package or device for preparing a composition, the kit comprising
6-24 mg/ml of type IV collagen;
0.04-0.15 M of sodium ions and/or 0.008-0.4 M of calcium ions; and
one or more crosslinking agents.

51. Use of a kit, package or device comprising type IV collagen, sodium ions, calcium ions, and one or more crosslinking agents, for preparing a composition comprising:
6-24 mg/ml of the type IV collagen;
0.04-0.15 M of the sodium ions and/or 0.008-0.4 M of the calcium ions; and
the one or more crosslinking agents.

52. The kit, package or device of claim 50 or the use of claim 51, wherein the composition comprises 0.01-0.1 mg riboflavin.

53. The kit, package or device of claim 50 or claim 52 or use of claim 51 or claim 52, wherein the one or more crosslinking agents are capable of activation by light.

54. The kit, package or device or the use of claim 53, wherein the light comprises UV light, blue light, green light or white light.

55. The kit, package or device of any one of claims 50 or 52 to 54 or the use of any one of claims 51 to 54, wherein the composition further comprises epithelial cells and/or endothelial cells.

56. The kit, package or device or the use of claim 55, wherein the epithelial cells are lens epithelial cells.

57. The kit, package or device or the use of claim 55, wherein the endothelial cells are corneal endothelial cells.

58. The kit, package or device of any one of claims 50 or 52 to 57 or the use of any one of claims 51 to 57, wherein the composition further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.
